(19) United States
(12) Patent Application Publication
(10) Pub. No.: US 2009/0132021 A1
(43) Pub. Date: May 21, 2009

(54) APPARATUS AND METHOD FOR DELIVERY, SPREADING AND DOUBLE-ENDED FIXATION OF VASCULAR GRAFTS

(76) Inventors: Edward G. Shifrin, Raanana (IL); Gennady S. Nickelshpur, Haifa (IL); Arkady S. Nickelshpur, Herzeliya (IL); Wesley S. Moore, Los Angeles, CA (US)

Correspondence Address: OSTROLENK FABER GERB & SOFFEN, 1180 AVENUE OF THE AMERICAS, NEW YORK, NY, 100368403, US

(21) Appl. No.: 12/159,662

(22) PCT Filed: Jan. 1, 2006
(86) PCT No.: PCT/IL06/00001

§ 371 Date: Nov. 12, 2008

(30) Foreign Application Priority Data
Feb. 1, 2005 (IL) 166648
Aug. 1, 2005 (IL) 170003

(51) Int. Cl. A61F 2/06; A61B 19/00

(52) U.S. Cl. 623/1.11; 128/898

(57) ABSTRACT

The present invention relates to medical techniques, more particularly to methods and apparatus for delivery and double-ended fixation of vascular prosthesis—grafts or stent-grafts to blood vessel walls in direction from inside these vessels to their outer surface. The apparatus comprises a tubular body with two expandable working heads at the free end, means for securing thereon the prosthesis end. The heads have eight cartridges each, and each of these cartridges is provided with one basic and one standby fastener means, substantially U-shaped staples. A control mechanism is provided consisting of two control modules joined together. The apparatus is also provided with means for positioning inside a blood vessel. There is further proposed a method for delivery and double-ended fixation of an endovascular prosthesis to a blood vessel wall.

### (30) PRIORITY DATA

IL 166648 1 Feb. 2005 (01.02.2005)
IL 170003 1 Aug. 2005 (01.08.2005)

This application is related to Patent Cooperation Treaty (“PCT”) patent applications PCT/IL2005/000115, filed Feb. 1, 2005, PCT/IL2005/000188, fled Feb. 15, 2005 and PCT/IL2005/001217, filed Nov. 17, 2005, as well as continuation-in-part Israel patent applications IL 166648, filed Feb. 1, 2005, IL 168993, filed Jun. 2, 2005, IL 169210, filed Jun. 16, 2005 and IL 170003, Filed Aug. 1, 2005.

### CROSS-REFERENCE TO RELATED APPLICATIONS

All our earlier patent applications relate to medicine, in particular, to vascular surgery. In all our earlier patent applications, other than IL 166648, filed Feb. 1, 2005, IL 168993, filed Jun. 2, 2005 IL 169210, filed Jun. 16, 2005 and IL 170003, Filed August 1, there are described apparatus and methods for delivery of vascular grafts or stent-grafts inside a blood vessel, substantially the aorta, and for their suturing by one end, which allows to accomplish surgical treatment of such diseases as aorta aneurysm. These methods and apparatus allow to prevent rupture of an aorta aneurysm by locating and suturing inside this aorta a synthetic prosthesis—graft or stent-graft. In patent applications IL 166648, filed Feb. 1, 2005, IL 168993, filed Jun. 2, 2005 and IL 170003, Filed August 1, there are described an apparatus and method for grafts or stent-grafts delivery inside the aorta and their alternate suturing by both ends. All these apparatus and methods have been developed on the basis of prior art and background described in detail in our above mentioned PCT and Israel patent applications.

At the same time, in performing surgical operations for preventing aorta rupture, in particular, of the abdominal aorta section, as well as in performing surgical operations for cleaning blood vessels from calcium patches and other thrombotic deposits using apparatus of “Rotablater” type, it would be appropriate to apply thin-wall or superthin-wall grafts—about 0.1 mm or even less, which might be spread, in particular, over cleaned inner surfaces of blood vessels to prevent formation of new thrombi and deposits. This raises the question of delivering the thin-wall graft inside a blood vessel and successively performing the steps necessary for suturing this graft by one end, its stretching or spreading along the blood vessel lumen, as well as suturing the graft by its second end.

An object of the present invention is to develop a new method and apparatus providing delivery and double-ended fixation of implanted endovascular prosthesis—grafts or stent-grafts, in corresponding blood vessels.

Another object of the present invention is to develop a system of apparatus assembled from a kit of standardized parts (similar to a construction kit for children) which allows to assemble fast an apparatus adapted to the anthropometric conditions of a specific patient and intended for performing a surgical operation to deliver a graft or stent-graft of a specific type and size and double-ended fixation of the latter to the a blood vessel wall.

A third object of the present invention is to develop a new method of endovascular thin wall grafts spreading and double-ended fixation to blood vessel walls.

### SUMMARY OF THE INVENTION

The subject-matters of the present invention are an apparatus and method for delivery and double-ended fixation of endovascular grafts or stent-grafts to blood vessel walls. The proposed apparatus comprises a tubular body, two expandable working heads with cartridges intended for location of fastener means, with means for securing the ends of an endovascular graft or stent-graft on these heads, as well as a control mechanism containing two control modules joined together via a connecting means.

The apparatus tubular body is configured for positioning within a vessel. This tubular body is rigid in longitudinal direction and flexible in lateral direction, at one its end there are located two working heads, and at its second end—the control mechanism.

The apparatus has two expandable working heads, each with cartridges intended for location of fastener means. These working heads are located at the free end of tubular body and made expandable to keep both ends of a delivered graft or stent-graft in contact with the blood vessel inner surface at the moment of their mutual fixation.

Each of the two working heads contains a cartridge group including substantially eight cartridges which are incorporated in the construction of a corresponding working head. All the cartridges are unified and interchangeable, and each of these cartridges is intended for location at least of two fastener means. All the cartridges are provided with means for retaining therein fastener means, for imparting thereto progressive motion, for forming fastener means during their extension from these cartridges, as well as for storing and feeding standby fastener means. Both working heads are also provided with means for securing thereon the ends of an endovascular graft.

Control mechanism of the proposed apparatus is provided with a first control module and second control module joined together via a connecting means. The control mechanism is located at the tubular body end opposite to the working heads and associated with these working heads via the tubular body. The first control module is located at the free end of this tubular body, and the second control module is located on the tubular body near the first module, between the latter and the second expandable working head. The first control module serves for operation of first working head, and second control module serves for operation of second working head. Control modules are joined together via a connecting means located between them on the tubular body.

The proposed apparatus have two embodiments. In the first embodiment this apparatus may be used for delivery and double-ended fixation of endovascular grafts or stent-grafts. The control mechanism of the apparatus have a rigid fixation of modules relative to one another.

In the second embodiment the apparatus may be used for delivery, spreading and double-ended fixation of thin wall endovascular grafts. For this purpose the second working head, which is secured near the free end of tubular body, some distance away from the first working head, aligned with the latter and capable of reciprocation relative thereto, and a connecting means, located on the tubular body between the first and second control modules capable of providing reciprocation of second control module and second working head rigidly attached thereto relative to the first control module. As a result, a surgical operation may be performed for thin wall graft delivery, spreading within the blood vessel and fixing both its ends to the walls of a blood vessel wall, substantially the aorta, from inside the aorta.

The first expandable working head contains a first pressure bush, first and second bearing bushes, arranged in succession one after another beginning from the free end of this working head and in alignment with the tubular body, as well as a first axial tube rigidly connected with the first bearing bush and passing via a through axial hole in second bearing bush.

First expandable working head is provided with eight cartridges which are pivotally secured by one their ends on first bearing bush and pivotally connected by their other ends with bearing levers pivotally mounted on second bearing bush.

Second expandable working head located some distance away from first working head contains a third and fourth bearing bushes and second pressure bush which are arranged successively one after another beginning from the free end of this working head facing the first expandable working head. Second head also has a second axial tube rigidly connected with third bearing bush and passing via through axial holes in fourth bearing bush and in second pressure bush. Third and fourth bearing bushes and second pressure bush are aligned with tubular body. Second working head is also provided with eight cartridges secured by one their ends on fourth bearing bush and pivotally connected by their other ends with bearing levers which are pivotally mounted on third bearing bush.

First and second working heads are provided with means for securing a delivered graft or stent-graft located on bearing levers and shaped substantially as radial tongues located at one of the ends of these bearing levers, near their pivotal connections with corresponding cartridges.

Each of the cartridges contains a means for retaining therein at least one basic fastener means, substantially U-shaped staple. This means for retaining is located along the cartridge and contains substantially a sliding lid.

Each of the apparatus cartridges includes at least one means for imparting progressive motion to the fastener means, substantially U-shaped staple, and this means contains a flexible pusher, rigid in longitudinal direction and flexible in lateral direction. All flexible pushers are associated by one their end with first or second pressure bush of first or second expandable working head respectively and located under the sliding lid to reciprocate in the clearance between the cartridge bottom and this sliding lid.

Each of the apparatus cartridges contains a means for forming fastener means located in this cartridge near one of its ends, as well as at least one basic fastener means, substantially U-shaped staple, located in this cartridge substantially along its longitudinal axis, with free pointed ends facing this means for forming. The U-shaped staple is located in the cartridge so as to progressively extend therefrom and be formed during this extension.

Each cartridge has a bottom and sliding lid and contains a means for storing at least one standby fastener means, substantially standby U-shaped staple. This means for storing contains a slot in cartridge bottom wherein at least one spring-loaded standby U-shaped staple is located. The standby staple is located in this slot substantially along the longitudinal cartridge axis, with free pointed ends facing the means for forming. This standby U-shaped staple is located in the slot of this cartridge bottom substantially under the flexible pusher so as to extend from this slot by action of spring and at partial removal of this flexible pusher from the clearance between the cartridge bottom and sliding lid. In the most preferred embodiment the slot in each cartridge bottom is a through one, and the standby U-shaped staple is, located in this slot immediately under the flexible pusher and spring-loaded on the opposite side.

Each of the apparatus cartridges has a body with a bottom provided with a recess for location at least of one basic fastener means, substantially U-shaped staple, and a through slot for location at least of one standby fastener means, substantially standby spring-loaded U-shaped staple, slots for mounting the sliding lid, as well as with a longitudinal duct for location at least of one flexible pusher. Therewith, the basic U-shaped staple is located in bottom recess of cartridge body in frictional contact with the sliding lid, which prevents its dropping out of this recess, and standby U-shaped staple is located in the through bottom slot of cartridge body in frictional contact with the flexible pusher, which prevents its extension from this through slot before partial removal of this flexible pusher.

The body bottom of each cartridge has a recess at least for one basic fastener means, substantially U-shaped staple. This recess is symmetric about the longitudinal axis of this cartridge and parallel with its outer surface. The cartridge is provided with a means for forming U-shaped staple during its extension from the recess, and this means contains a curvilinear guiding surface connecting the bottom of this recess for U-shaped staple with the cartridge outer surface. The curvilinear guiding surface has forming grooves diverging at an acute angle from one another and from the cartridge longitudinal axis. The basic fastener means, substantially U-shaped staple, has pointed free ends and is located in body recess of this cartridge in such a way, that its pointed ends face corresponding forming grooves on curvilinear guiding surface.

Forming grooves diverging at an acute angle from one another and from the cartridge longitudinal axis provide helical oppositely directed curling of pointed ends of a corresponding U-shaped staple during its movement over the body recess and forming grooves of curvilinear guiding surface of this cartridge.

Each apparatus cartridge is shaped substantially as a polyhedral prism with two skewed side faces inclined at an acute angle to one another. The vertex of this acute angle lies on the longitudinal axis of expandable working head. All cartridges are provided with means for connection respectively with first or fourth bearing bush of first or second working head or with a corresponding bearing lever. These means contain arms with holes which project from both bases of the polyhedral prism and are integral with this polyhedral prism.

All U-shaped staples are made from one of the materials of a group including stainless steel, titanium and shape memory alloys.

The apparatus is additionally provided with a means for axial precision positioning containing a measuring scale on the tubular body surface, as well as with means for radial precision positioning containing markers—X-ray marks evenly applied on the upper surface of cartridges and serving for orientation of the apparatus working heads by their operation angle.

The control mechanism has a first and a second control modules joined together via a connecting means. First control module is located at the free end of tubular body opposite to first expandable working head, and second control module is rigidly connected with second expandable working head and located on tubular body near first control module, between the latter and second expandable working head. The connecting means located on tubular body between first and second control modules may provide either their rigid mutual fixation, or reciprocation of second control module and second working head rigidly attached thereto relative to first control module. This connecting means substantially contains a holder with a gear pivotally mounted therein and provided with a lock and rotary drive, as well as two toothed racks operably engaged with the gear, the first of the racks being rigidly connected with first control module, and the second rigidly connected with second control module. The connecting means is additionally provided with a handle and support for carrying the apparatus and its location on the operating table. It is also provided with the gear rotary drive which is substantially mechanic or electric.

First control module of control mechanism contains a hollow body having a first and second ends, as well as a slider enclosed in this body to reciprocate relative to the latter. The slider is provided with a holding handle rigidly attached thereto and functionally associated with the pivotal head located at the second end of this hollow body. This slider together with the pivotal head forms the first control means of first control module of the control mechanism.

First control module of the control mechanism further contains a first pressure handle serving as a second control means. This first pressure handle is pivotally attached to holding handle and has a short free end enclosed in the slider. The slider is rigidly connected with first axial tube serving as a first connecting means, and the short free end of pressure handle is functionally associated with a tie serving as a second connecting means of first control module of the control mechanism. First pressure handle is provided with a swing lock pivotally mounted on the slider, as well as with a flat return spring secured by one its end to this pressure handle, and by its second end—to the holding handle.

In the proposed apparatus first bearing bush of the first expandable working head is functionally connected via first connecting means with first control means of the first control module. First pressure bush is functionally connected via second connecting means with second control means of first control module of the control mechanism. Second bearing bush is rigidly connected with a distance tube located between first and second working heads. First connecting means and second connecting means are enclosed in the distance tube and tubular body concentrically with one another, with the distance tube and this tubular body.

Second control module of the control mechanism contains a hollow body with a handle rigidly attached thereto or integral therewith. This body has a first and second end, is rigidly connected by its first end via a retaining tube with fourth bearing bush of second working head and also includes a slider enclosed in the body to reciprocate relative to the latter. The slider is functionally associated with the pivotal head located at the second end of this hollow body and forms together with this pivotal head a first control means of second control module of the control mechanism.

The second control module also has a second pressure handle serving as its second control means and pivotally secured to the handle or hollow body. This second pressure handle has a short free end enclosed in the slider, and the latter is rigidly connected with a tubular tie serving as a third connecting means, and the short free end of pressure handle is functionally associated with a pressure tube serving as a fourth connecting means of the control mechanism and rigidly connected with the second pressure bush. Second pressure handle is provided with a swing lock pivotally mounted in the hollow body, as well as with a flat return spring secured by one its end to this second pressure handle, and by its other end to the hollow body handle.

Third bearing bush is connected via third connecting means with first control means of second control module of the control mechanism. Second pressure bush is functionally connected via fourth connecting means with second control means of second control module of the control mechanism, and fourth bearing bush is connected with the body of second control module via retaining tube. Third connecting means of second module is enclosed in the retaining tube, and this fourth connecting means of second module is located outside this retaining tube, and they are all arranged concentrically with one another and this retaining tube.

First and second connecting means connect first expandable working head with first control module of the control mechanism. They are arranged concentrically with one another within third connecting means, retaining tube and fourth connecting means which are also arranged concentrically with one another and link second working head with second control module of the control mechanism. The distance tube is located between first and second working heads outside the first and second connecting means and in alignment with third and fourth connecting means. The fourth connecting means located outside, concentrically with retaining tube, third connecting means, as well as with first and second connecting means, forms a tubular body configured for positioning within a vessel. This tubular body, together with connecting means enclosed therein, is rigid in longitudinal direction and flexible in lateral direction.

Another subject-matter of the present invention is a method for delivery and double-ended fixation of grafts or stent-grafts to the wall of a blood vessel from within the latter, comprising several successive steps.

At the first step, the graft or stent-graft is prepared for delivery, mounted and its both ends secured on the apparatus working heads. The graft or stent-graft is located between the working heads and crimped to a given outer diameter.

At the second stage, the apparatus for delivery and fixation is brought in operative position, inserted into a corresponding blood vessel, first expandable working head is brought to the securing area, and precision axial and radial positioning of the apparatus at a given blood vessel point is performed via a measuring scale on tubular body surface and via markers—X-ray contrast marks. Then first working head is deployed in such a way, that the first end of a delivered graft or stent-graft is in contact with the inner surface of this blood vessel at the moment of their mutual fixation, and the cartridges of this expandable working head are located near corresponding suturing points.

Thereupon U-shaped staples in the cartridges of first expandable working head are set in motion, which is accomplished by action of flexible pushers activated, in their turn, via first pressure bush, second connecting means and second control means of first module of the control mechanism, which is associated with these pushers. As a result, pointed ends of each of the U-shaped staples enter the forming grooves of curvilinear guiding surface of a corresponding removable cartridge, are curled therein, diverging in opposite directions from one another, pierce the prosthesis wall and surrounding blood vessel wall and return again over a spiral to the curvilinear guiding surface, repeating this rotation if permitted by the given staple length. As a result, the wall of this prosthesis first end gets sutured by wire spirals formed from U-shaped staples to a corresponding area of the blood vessel wall, and the U-shaped staples themselves entirely emerge from recesses of corresponding cartridges and are released from these cartridges, forming an oversew suture at the first end of a corresponding prosthesis.

Then the cartridges of first expandable working head are reloaded with standby U-shaped staples. This is accomplished by partial removal of flexible pushers activated, in their turn, via first pressure bush, second connecting means and second control means of first module of the control mechanism, which are associated with these pushers. As a result, each of the spring-loaded standby U-shaped staples emerges from the through slot of a corresponding cartridge, and then, at reverse motion of a corresponding flexible pusher, moves by its action into a recess wherein the basic U-shaped staple was located before. Now the first head of the apparatus for delivery and fixation is ready for another suturing.

Then, if another suturing is necessary, U-shaped staples in the cartridges of first expandable working head are set in motion. This is accomplished by action of flexible pushers activated, in their turn, via first pressure bush, second connecting means and second control means of first module of the control mechanism, which are associated with these pushers. As a result, pointed ends of each of the standby U-shaped staples enter the forming grooves of curvilinear guiding surface of a corresponding removable cartridge, are curled therein, diverging in opposite directions from one another, pierce the wall of the prosthesis first end and surrounding blood vessel wall and return again over a spiral to the curvilinear guiding surface, repeating this rotation if permitted by the given staple length. As a result, the wall of this prosthesis first end gets sutured again by wire spirals formed from standby U-shaped staples to a corresponding area of the blood vessel wall, and the standby U-shaped staples themselves entirely emerge from recesses of corresponding cartridges and are released from these cartridges, forming a second oversew suture at the first prosthesis end.

Then, if it is necessary, the prosthesis—a thin wall graft, sutured at one end, is spread. This is accomplished by rotation of the connecting means gear, which enables the second control module and second working head rigidly connected therewith to move along the apparatus tubular body some predesigned distance towards the first control means. The prosthesis secured by one its end on the blood vessel, and by the other—on the second working head, is spread over its whole length.

Then U-shaped staples in the cartridges of second working head are set in motion. This is accomplished by action of flexible pushers activated, in their turn, via second pressure bush, fourth connecting means and second control means of second module of the control mechanism, which are associated with these pushers. As a result, pointed ends of each of the U-shaped staples enter the forming grooves of curvilinear guiding surface of a corresponding removable surface, are curled therein, diverging in opposite directions from one another, pierce the prosthesis wall and surrounding blood vessel wall and return again over a spiral to the curvilinear guiding surface, repeating this rotation if permitted by the given staple length. As a result, the wall of this prosthesis second end gets sutured by wire spirals formed from U-shaped staples to a corresponding area of the blood vessel wall, and the U-shaped staples themselves entirely emerge from recesses of corresponding cartridges and are released from these cartridges, forming an oversew suture at the prosthesis second end.

Then, if necessary, the cartridges of second working head are reloaded with standby U-shaped staples. This is accomplished by partial removal of flexible pushers activated, in their turn, via second pressure bush, fourth connecting means and second control means of second module of the control mechanism. As a result, each of the spring-loaded standby U-shaped staples emerges from the through slot of a corresponding cartridge and then, at reverse motion of a corresponding flexible pusher, moves by its action into a recess where a basic U-shaped staples was located before. Now the second head of the apparatus for delivery and fixation is ready for another suturing.

If another suturing is necessary, standby U-shaped staples in the cartridges of second working head are set in motion. This is accomplished by action of flexible pushers activated, in their turn, via second pressure bush, fourth connecting means and second control means of second module of the control mechanism, which are associated with these pushers. As a result, pointed ends of each of the standby U-shaped staples enter the forming grooves of curvilinear guiding surface of a corresponding removable cartridge, are curled therein, diverging in opposite directions from one another, pierce the wall of this prosthesis second end and surrounding blood vessel wall and return again over a spiral to the curvilinear guiding surface, repeating this rotation if permitted by the given staple length. As a result, the wall of the prosthesis second end gets sutured again by wire spirals formed from standby U-shaped staples, to a corresponding area of the blood vessel wall, and the U-shaped staples themselves entirely emerge from recesses of corresponding cartridges and are released from these cartridges, forming a second oversew suture at the prosthesis second end.

Then the second end of a delivered and sutured graft is separated from the apparatus for delivery and fixation, both its expandable heads are returned to the original position, this apparatus is set in inoperative position and removed from the graft and from the blood vessel.

### BRIEF DESCRIPTION OF THE DRAWINGS

The invention will now be described in conjunction with the following drawings in which like reference numerals designate like elements and wherein:

FIG. 1 shows a general view of the proposed apparatus;

FIG. 2 shows the proposed apparatus during graft suturing;

FIG. 3 shows a general view of first and second working heads of the apparatus with a graft secured thereon at the moment of suturing the graft first end (first working head deployed);

FIG. 4 shows a general view of the second bearing bush;

FIG. 5 shows the general view of a cartridge in assembly with a sliding lid;

FIG. 6 shows the general view of a cartridge with arms for its securing and with a through slot in the bottom;

FIG. 7 shows a cartridge as viewed from the means for forming the staple;

FIG. 8 shows the general view of a cartridge as viewed from its bottom and through slot;

FIG. 9, 10 show cartridge views with diagrams of location of a basic and standby U-shaped staples;

FIG. 11 shows the apparatus control mechanism, and

FIG. 12 shows an operation diagram of the proposed apparatus.

### DETAILED DESCRIPTION OF PREFERRED EMBODIMENTS

The preferred embodiments of the present invention are described below. The inventors of the present subject matter contemplate that the embodiments described herein are capable of use in the repair of other vessels and in other procedures. Thus, it is intended that the present invention cover the modifications and variations of the invention, provided they come within the scope of the appended claims and their equivalents.

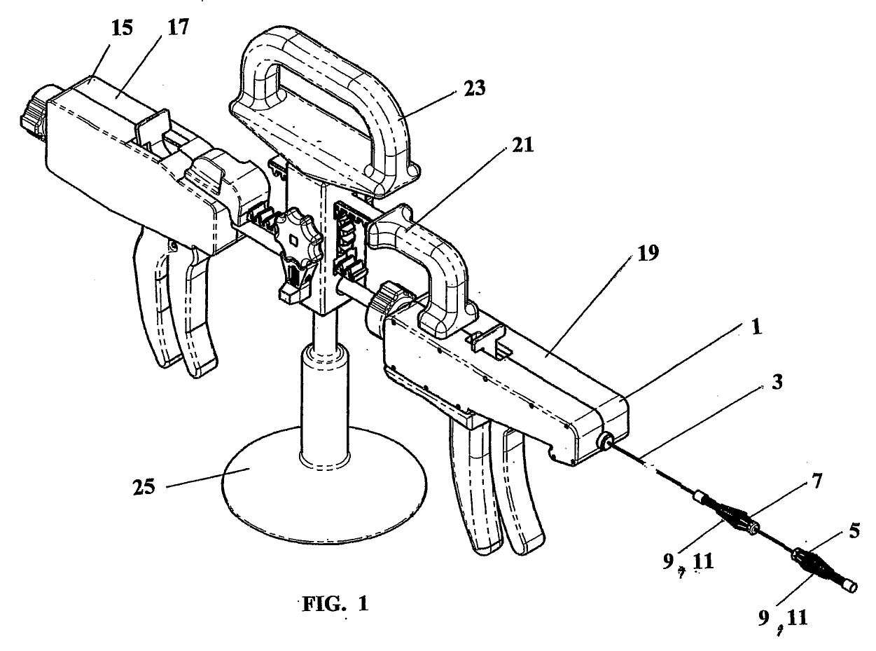

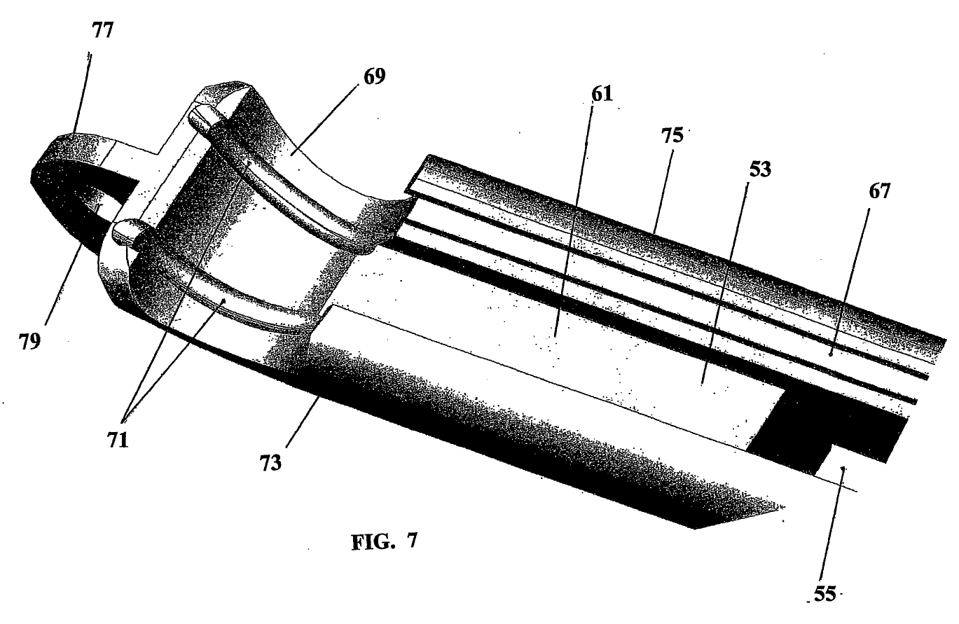

The most preferred embodiments of an apparatus, according to the present invention, are shown in drawing FIGS. 1-12.

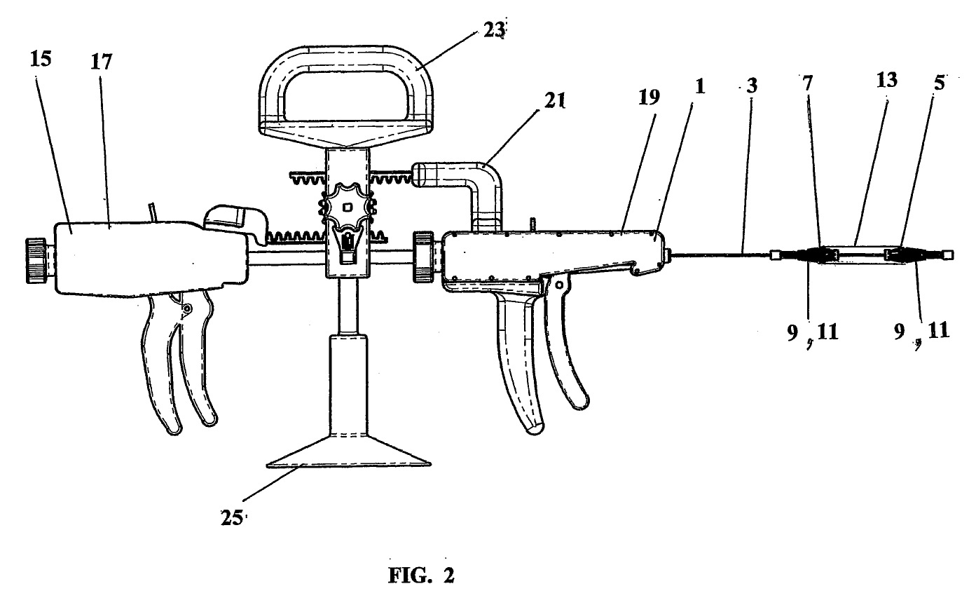

Proposed apparatus 1 (FIG. 1) comprises a tubular body 3, two expandable working heads 5 and 7 with cartridges 9 intended for location of fastener means, substantially U-shaped staples 11, as well as means for securing the ends of endovascular graft 13 on these heads (FIG. 2). Apparatus 1 is provided with a control mechanism 15 including a first control module 17, second control module 19, connecting means 21 joining them together with a crying handle 23 and support 25.

Tubular body 3 of apparatus 1 (FIG. 1) is configured for positioning within a vessel. Tubular body 3 is rigid in longitudinal direction and flexible in lateral direction, expandable working heads 5 and 7 are located at one its end (FIG. 1, 2), and at the second end—control mechanism 15.

Working heads 5 and 7 are located at the free end of tubular body 3 (FIG. 1, 2) and are made expandable to keep both ends of a delivered graft or stent-graft 13 in contact with the inner surface of a blood vessel at the moment of their mutual fixation. Each of working heads 5 and 7 contains a group of cartridges, including substantially eight cartridges 9 each, these cartridges being incorporated in the construction of a corresponding working head 5 or 7. All cartridges 9 are unified and interchangeable and each of them is intended for location at least of two fastener means—U-shaped staples 11. All cartridges 9 are provided with means for retaining therein fastener means, for imparting progressive motion thereto, for forming fastener means—U-shaped staples 11 during their extension from cartridges 9, as well as for storing and feeding standby fastener means.

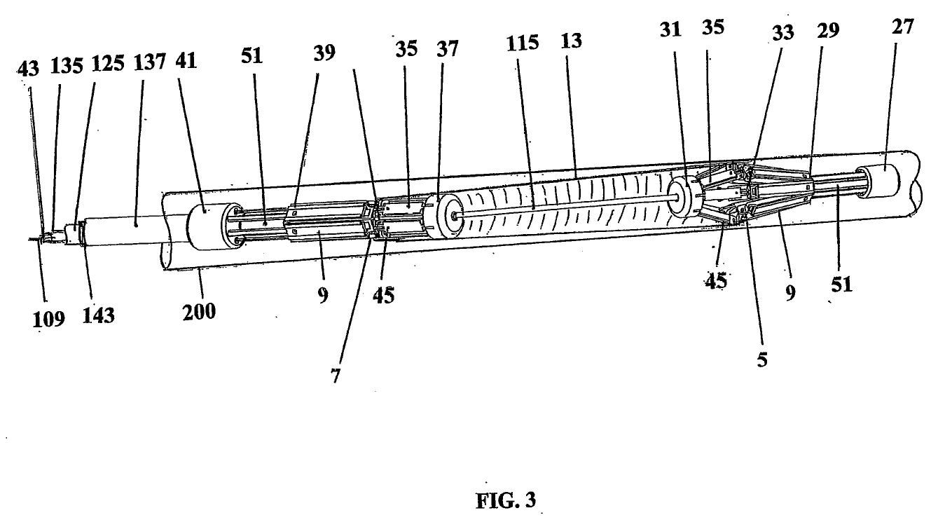

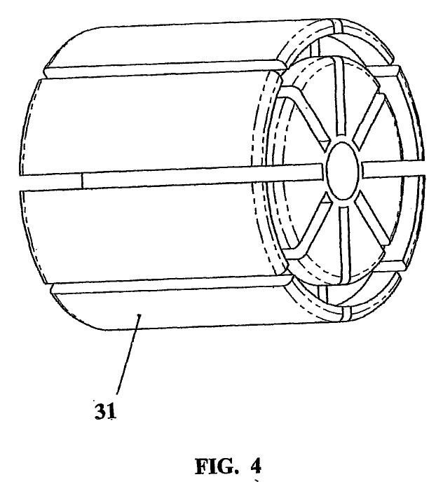

First expandable working head 5 (FIG. 3) contains a first pressure bush 27, first and second bearing bushes 29 and 31, which are arranged in succession one after another beginning from the free end of this working head 5 and in alignment with tubular body 3, as well as a first axial tube 33 rigidly connected with first bearing bush 29 and passing via through axial hole in second bearing bush 31. Second bearing bush 31 is shown in more detail in FIG. 4.

First expandable working head 5 (FIG. 3) is provided with eight cartridges 9 pivotally mounted by one their ends on first bearing bush 29 and pivotally connected by their other ends with bearing levers 35, pivotally mounted on second bearing bush 31.

Second expandable working head 7 (FIG. 3) located some distance away from first working head 5, contains a third and fourth bearing bushes, 37 and 39 respectively, and second pressure bush 41, which are arranged in succession beginning from the free end of this working head 7 facing first expandable working head 5. Second head 7 also has a second axial tube 43 rigidly connected with third bearing bush 37 and passing via through axial holes in fourth bearing bush 39 and in second pressure bush 41. Third and fourth bearing bushes 37, 39 and second pressure bush 41 are aligned with tubular body 3. Second working head 7 (FIG. 3) is also provided with eight cartridges 9, mounted by one their ends on fourth bearing bush 39 and pivotally connected by their other ends with bearing levers 35, pivotally mounted on third bearing bush 37.

Both working heads 5 and 7 are also provided with means for securing thereon the ends of endovascular graft or stent-graft 13 located on bearing levers 35 and shaped substantially as radial tongues 45 located at one of the ends of these bearing levers 35, near their pivotal connections with corresponding cartridges 9 (FIG. 3).

Control mechanism 15 of apparatus 1 is provided with a first control module 17 and second control module 19 linked together via a connecting means 21. Control mechanism 15 (FIG. 1, 2) is located at the end of tubular body 3 opposite to working heads 5 and 7 and is associated thereby with these working heads 5 and 7. First control module 17 is located at the free end of tubular body 3, and second control module 19 is disposed on tubular body 3 near first module 17, between the latter and second expandable working head 7. First control module 17 serves to operate first working head 5, and second control module 19 serves to operate second working head 7. Control modules 17 and 19 are joined together via a connecting means 21 located between them on tubular body 3. It provides both rigid fixation of modules 17 and 19 relative to one another, and reciprocation of second control module 19 and second working head 7 rigidly attached thereto relative to first control module 17 (FIG. 1, 2).

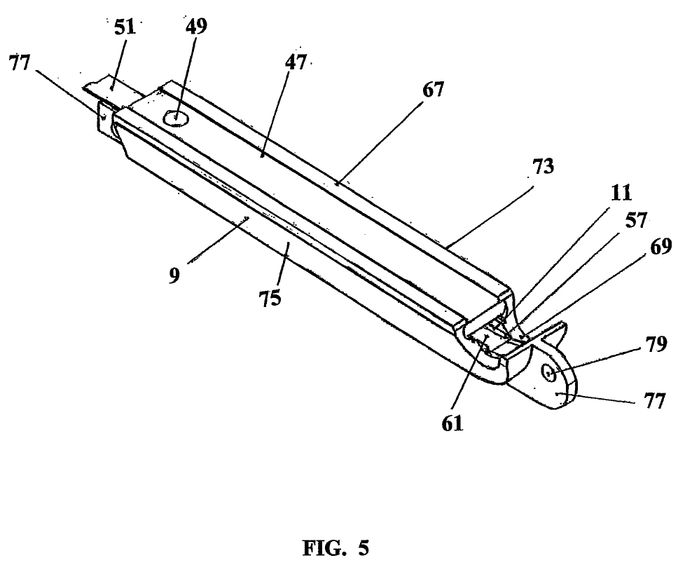

Each cartridge 9 (FIG. 5) contains a means for retaining therein at least one basic fastener means—U-shaped staple 11. This means for retaining is located along cartridge 9 and contains a sliding lid 47 with a hole 49 for its opening.

Each cartridge 9 (FIG. 3, 5) of apparatus 1 includes at least one means for imparting progressive motion to a fastener means—U-shaped staple 11, and this means contains a flexible pusher 51, rigid in longitudinal direction and flexible in lateral direction. All flexible pushers 51 are associated by one end respectively with first or second pressure bush 27 or 41 of first or second expandable working head 5 or 7 and located under sliding lid 47 of cartridge 9 to reciprocate in the clearance between the bottom of cartridge 9 and this sliding lid 47.

Each cartridge 9 (FIG. 5) of apparatus 1 contains a means for forming fastener means located in this cartridge 9 near one of its ends, as well as at least one basic fastener means—U-shaped staple 11 disposed in this cartridge 9 substantially along its longitudinal axis, with free pointed ends facing this means for forming. U-shaped staple 11 is disposed in cartridge 9 so as to progressively extend therefrom and be formed during this extension.

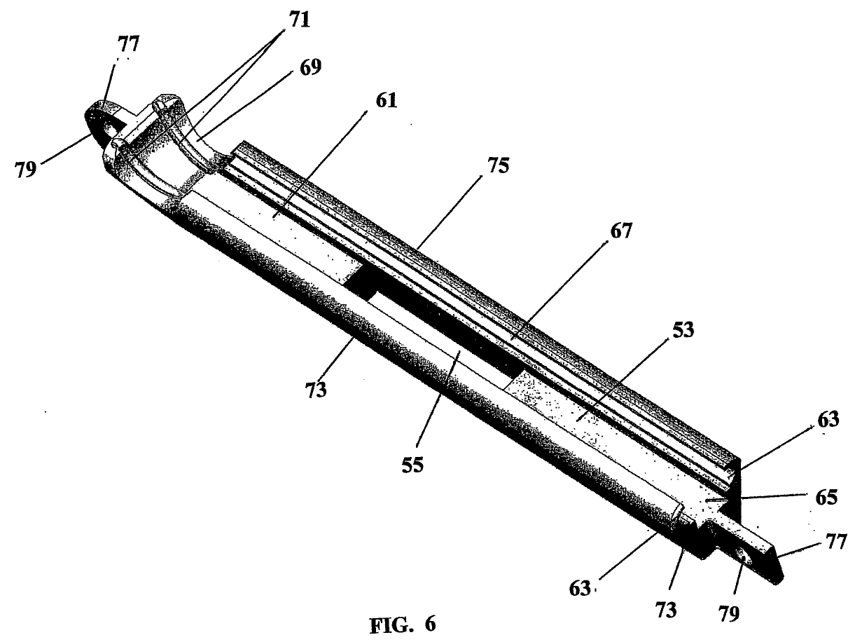

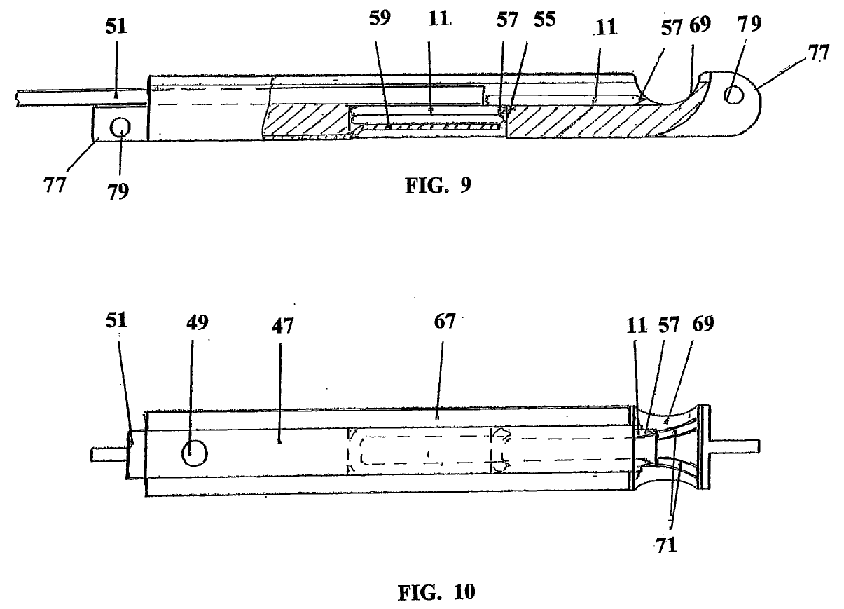

All cartridges 9 (FIG. 5, 6) have a bottom 53 and sliding lid 47 and contain a means for storing at least one standby fastener means, containing a standby U-shaped staple 11. This means for storing contains a slot 55 in the bottom 53 of cartridge 9 (FIG. 6), wherein at least one spring-loaded standby U-shaped staple 11 is located. Standby staple 11 is disposed in this slot 55 substantially along the longitudinal axis of cartridge 9, with free pointed ends 57 facing the means for forming. Standby U-shaped staple 11 is disposed in slot 55 of bottom 53 of cartridge 9 (FIG. 9) substantially under flexible pusher 51 to extend from this slot 55 by action of spring 59 and at partial removal of flexible pusher 51 from the clearance between bottom 53 of cartridge 9 and its sliding lid 47. In the most preferred embodiment (FIG. 6-10) slot 55 in bottom 53 of each cartridge 9 is a through one, and standby U-shaped staple 11 is located in this slot 55 immediately under flexible pusher 51 and loaded with spring 59 on the opposite side.

Each cartridge 9 (FIG. 5-10) of apparatus 1 has a body with a bottom 53 with recess 61 for location at least of one U-shaped staple 11 and through slot 55 for location at least of one more standby fastener means—a like spring-loaded U-shaped staple 11, slots 63 for mounting sliding lid 47, as well as with a longitudinal duct 65 for location at least of one flexible pusher 51. Basic U-shaped staple 11 is disposed in recess 61 of bottom 53 of cartridge 9 in frictional contact with sliding lid 47, which prevents its dropping out of this recess 61, and standby spring-loaded U-shaped staple 11 is disposed in through slot 55 of bottom 53 of cartridge 9 in frictional contact with flexible pusher 51, which prevents its extension from this through slot 55 before partial removal of flexible pusher 51.

Bottom 53 of each cartridge 9 (FIG. 5-10) has a recess 61 for a basic fastener means—U-shaped staple 11. This recess 61 is symmetric about the longitudinal axis of cartridge 9 and parallel with its outer surface 67 (FIG. 5-7). Cartridge 9 is provided with a means for forming U-shaped staple 11 during its extension from recess 61, and this means contains a curvilinear guiding surface 69 connecting the bottom of recess 61 for U-shaped staple 11 with outer surface 67 of cartridge 9. Curvilinear guiding surface 69 has forming grooves 71 (FIG. 5-7 and 10), diverging at an acute angle from one another and from the longitudinal axis of cartridge 9. The basic fastener means—U-shaped staple 11, has pointed free ends 57 and is located in recess 61 of this cartridge 9 in such a way, that its pointed ends 57 face corresponding forming grooves 71 of curvilinear guiding surface 69.

Forming grooves 71 diverging at an acute angle from one another and from the longitudinal axis of cartridge 9 (FIG. 5-10) provide helical oppositely directed curling of pointed ends 57 of a corresponding U-shaped staple 11 during its movement over recess 61 and forming grooves 71 of curvilinear guiding surface 69 of cartridge 9.

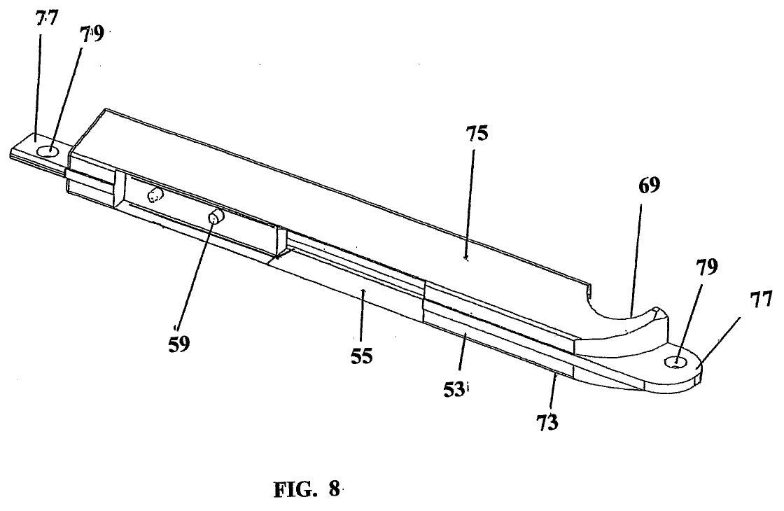

All cartridges 9 of apparatus 1 are substantially shaped as a polyhedral prism with two skewed side faces 73, 75, inclined at an acute angle to one another (FIG. 5-10). The vertex of this acute angle lies on the longitudinal axis of expandable working head 5 or 7. All cartridges 9 are provided with means for connection respectively with first 29 or fourth 39 bearing bushes of first or second working head 5, 7 or with a corresponding bearing lever 35. These means contain arms 77 with holes 79 (FIG. 8), which project from both bases of the polyhedral prism and are integral with this polyhedral prism.

All U-shaped staples 11 are made from one of the materials of a group including stainless steel, titanium and shape memory alloys.

Apparatus 1 is additionally provided with a means for axial precision positioning containing a measuring scale on the surface of tubular body 3, as well as with means for radial precision positioning containing markers—X-ray contrast marks evenly applied on the outer surface of cartridges 9 and serving for orientation of working heads 5 and 7 of apparatus 1 by their operation angle (not shown in the drawings).

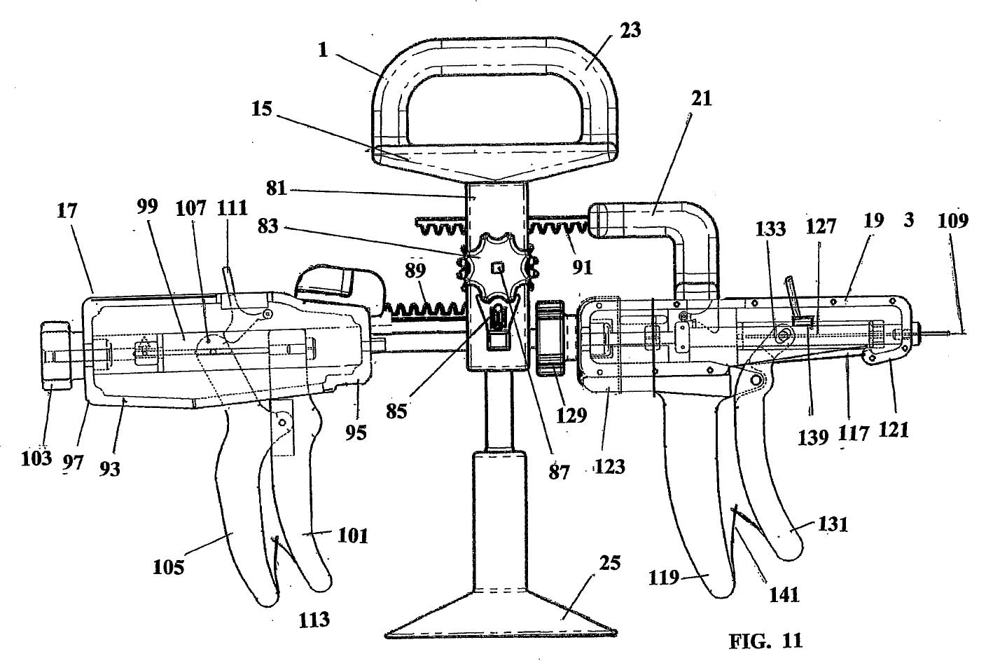

Control mechanism 15 includes first and second control modules 17 and 19 joined together via a connecting means 21. Connecting means 21 (FIG. 11) is located on tubular body 3 between first and second control modules 17 and 19, and provides both their rigid mutual fixation, or reciprocation of second control module 19 and second working head 7 rigidly attached thereto relative to first control module 17. This connecting means 21 (FIG. 11) contains a holder 81 with a gear 83 pivotally mounted therein and provided with a lock 85 and a mechanic or electric rotary drive connected with shaft 87, as well as two toothed racks 89 and 91 functionally engaged with gear 83, the first whereof—89 is rigidly connected with first control module 17, and the second, 91 is rigidly connected with second control module 19. The connecting means is additionally provided with handle 23 and support 25 for carrying apparatus 1 and its location on the operation table.

First control module 17 of control mechanism 15 contains a hollow body 93 (FIG. 11) having a first and second ends 95 and 97, as well as slider 99 enclosed in this body 93 to reciprocate relative to the latter. Slider 99 is provided with a holding handle 101 rigidly attached thereto and functionally associated with pivotal head 103 located at second end 97 of this hollow body 93. This slider 99 together with pivotal head 103 forms the first control means of first control module 17 of control mechanism 15.

First control module 17 of control mechanism 15 further contains a first pressure handle 105 serving as the second control means. This first pressure handle 105 is pivotally secured to holding handle 101 and has a short free end 107 enclosed in slider 99. Slider 99 is rigidly connected with first axial tube 33 (FIG. 3) serving as a first connecting means, and the short free end 107 of pressure handle 105 is functionally associated with tie 109 (FIG. 3, 11) serving as a second connecting means of first control module 17 of control mechanism 15. First pressure handle 105 is provided with a swing lock 111 pivotally mounted on slider 99, and a flat return spring 113 secured by one its end to this pressure handle 105, and by its second end—to holding handle 101.

In proposed apparatus 1 first bearing bush 29 of first expandable working head 5 is functionally connected via first connecting means—first axial tube 33 with first control means of first control module 17 (FIG. 3,11). First pressure bush 27 is functionally connected via second connecting means—tie 109 with the second control means of first control module 17 of control mechanism 15. Second bearing bush 31 is rigidly connected with distance tube 115 disposed between first and second working heads 5 and 7. First connecting means and second connecting means are enclosed in distance tube 115 and tubular body 3 concentrically with one another, distance tube 115 and this tubular body 3.

Second control module 19 of control mechanism 15 (FIG. 3, 11) contains a hollow body 117 with handle 119 rigidly attached thereto or integral therewith. This body 117 has a first and second ends 121 and 123, is rigidly connected by its first end via holding tube 125 (FIG. 3) with second working head 7 and includes a slider 127 enclosed in body 117 to reciprocate relative to the latter. Slider 127 is functionally associated with pivotal head 129 mounted on second end 123 of this hollow body 117 and forms together with this pivotal head 129 a first control means of second control module 19 of control mechanism 15.

Second control module further has a second pressure handle 131 serving as its second control means and pivotally attached to handle 119 or to hollow body 117. This second pressure handle 131 has a short free end 133 enclosed in slider 127. Slider 127 is rigidly connected with tubular tie 135 (FIG. 3) serving as a third connecting means, and the short free end 133 of pressure handle 131 is functionally associated with pressure tube 137 serving as a fourth connecting means of control mechanism and rigidly connected with second pressure bush 41. Second pressure handle 131 is provided with a swing lock 139 pivotally mounted in hollow body 117, as well as a flat return spring 141 secured by one its end to this second pressure handle 131, and by its second end to handle 119 of this body 117.

Third bearing bush 37 is connected via third connecting means—tubular tie 135 with first control means of second control module 19 of control mechanism 15. Second pressure bush 41 is functionally connected via fourth connecting means—pressure tube 137 with second control means of second control module 19 of control mechanism 15. Fourth bearing bush 39 is connected with the body of second control module 19 via retaining tube 143. Third connecting means of second module 19—tubular tie 135 is enclosed in retaining tube 143, and said fourth connecting means—pressure tube 137 is located outside this retaining tube 143 and they are all arranged concentrically with one another and with this retaining tube 143.

First and second connecting means—first axial tube 33 and tie 109, link first expandable working head 5 with first control module 17 of control mechanism 15. They are arranged concentrically with one another and enclosed in third connecting means—tubular tie 135, retaining tube 143 and fourth connecting means—pressure tube 137, which are also arranged concentrically with one another and link second working head 7 with second control module 19 of control mechanism 15. Distance tube 115 is located between first and second working heads 5 and 7, outside first and second connecting means and in alignment with third and fourth connecting means. Fourth connecting means—pressure tube 137, is located outside, concentrically with retaining tube 143, third connecting means—tubular tie 135, as well as with first and second connecting means—first axial tube 33 and tie 109, forming a tubular body 3 configured for positioning within a vessel. This tubular body 3, together with connecting means enclosed therein, is rigid in longitudinal direction and flexible in lateral direction.

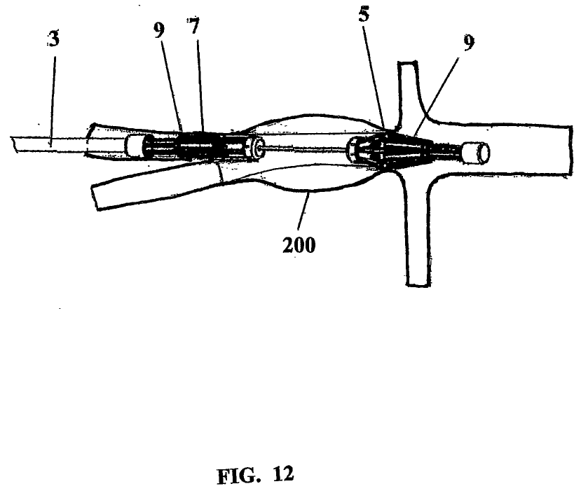

The proposed apparatus 1 operates on the basis of the proposed method for delivering prosthesis—graft or stent-graft 13 inside a blood vessel, substantially aorta 200, and fixation of prosthesis—graft or stent-graft 13 to the wall of aorta 200, from inside the latter (FIG. 12). The proposed method comprises several successive steps. At the first step graft or stent-graft 13 is prepared for delivery, mounted, and its both ends are secured on working heads 5 and 7 of apparatus 1, graft or stent-graft 13 is located between working heads 5 and 7 and crimped to a given outer diameter.

At the second step, apparatus 1 for delivery and fixation is set in operative position, inserted into a corresponding blood vessel, in particular, aorta 200, first expandable working head 5 is brought to the securing area, precision axial and radial positioning of apparatus 1 is performed at a given point of the blood vessel—aorta 200 via a measuring scale on the surface of tubular body 3 and via markers—X-ray contrast marks (FIG. 12). Then first working head 5 is deployed in such a way, that the first end of a delivered prosthesis—graft or stent-graft 13 is in contact with the inner surface of the blood vessel—aorta 200 at the moment of their mutual fixation, and cartridges 9 of this expandable working head 5 are located near corresponding suturing points.

Then U-shaped staples 11 in cartridges 9 of first expandable working head 5 are set in motion, which is accomplished by action of flexible pushers 51 activated, in their turn, via first pressure bush 27, second connecting means—tie 109 and second control means—pressure handle 105 of first module 17 of control module 15, which are associated with these pushers. As a result, pointed ends 57 of each U-shaped staple 11 enter forming grooves 71 of curvilinear guiding surface 69 of a corresponding removable cartridge 9, are curled therein, diverging in opposite directions from one another, pierce the wall of prosthesis—graft or stent-graft 13 and surrounding wall of aorta 200 and return again over a spiral to curvilinear guiding surface 69, repeating this rotation if permitted by the given length of staples 11. As a result, the wall of first end of prosthesis—graft or stent-graft 13 gets sutured by wire spirals formed from U-shaped staples 11, to a corresponding wall area of aorta 200, and U-shaped staples 11 themselves entirely emerge from recesses 61 of corresponding cartridges 9 and are released from these cartridges 9, forming an oversew suture at the first end of prosthesis—graft or stent-graft 13 (FIG. 12).

Then cartridges 9 of first expandable working head 5 are reloaded with standby U-shaped staples 11. This is accomplished by partial removal of flexible pushers 51 activated, in their turn, via first pressure bush 27, second connecting means—tie 109 and second control means—pressure handle 105 of first module 17 of control mechanism 15, which are associated with these pushers. As a result, each spring-loaded standby U-shaped staple 11 emerges from through slot 55 of a corresponding cartridge 9, and then, at reverse motion of a corresponding flexible pusher 51, moves by its action into recess 61, wherein the basic U-shaped staple 11 was located before. Now, first head 5 of apparatus 1 for delivery and fixation is ready for another suturing.

Then, if another suturing is necessary, standby U-shaped staples 11 in cartridges 9 of first expandable working head 5 are set in motion. This is accomplished by action of flexible pushers 51 activated, in their turn, via first pressure bush 27, second connecting means—tie 109 and second control means—pressure handle 105 of first module 17 of control mechanism 15. As a result, pointed ends 57 of each standby U-shaped staple 11 enter forming grooves 71 of curvilinear guiding surface 69 of a corresponding removable cartridge 9, are curled therein, diverging in opposite directions from one another, pierce the wall of the first end of prosthesis—graft or stent-graft 13 and surrounding wall of aorta 200 and return again over a spiral to curvilinear guiding surface 69, repeating this rotation if permitted by the given length of staples 11. As a result, the wall of the first end of prosthesis—graft or stent-graft 13 gets sutured again by wire spirals formed from standby U-shaped staples 11, to a corresponding wall area of aorta 200, and U-shaped staples 11 themselves entirely emerge from recesses 61 of corresponding cartridges 9 and are released from these cartridges 9, forming a second oversew suture at the first end of prosthesis—graft or stent-graft 13 (FIG. 12).

Then prosthesis—if it is thin wall graft 13, which sutured at one end, is spreading, as it is necessary. This is accomplished by rotation of gear 83 of connecting means 21, which enables second control module 19 and second working head 7 rigidly connected therewith to move along tubular body 3 of apparatus 1 a certain, predetermined distance towards first control module 17. Graft 13 secured by one end on the blood vessel—aorta 200, and by the other end—on second working head 7, is being spread over its entire length (FIG. 11, 12).

Then U-shaped staples 11 in cartridges 9 of second working head 7 are set in motion. This is accomplished by action of flexible pushers 51 activated, in their turn, via second pressure bush 41, fourth connecting means—pressure tube 137 and second control means—pressure handle 131 of second module 19 of control mechanism 15. Thereby, pointed ends 57 of each U-shaped staple 11 enter forming grooves 71 of curvilinear guiding surface 69 of a corresponding removable cartridge 9, and curled therein, diverging in opposite directions from one another, pierce the wall of prosthesis—graft or stent-graft 13 and surrounding wall of aorta 200 and return again over a spiral to curvilinear guiding surface 69, repeating this rotation if permitted by the given length of staples 11. As a result, the wall of the second end of prosthesis—graft or stent-graft 13 gets sutured by wire spirals formed from U-shaped staples 11, to a corresponding wall area of aorta 200, and U-shaped staples 11 themselves entirely emerge from recesses 61 of corresponding cartridges 9 and are released from these cartridges 9, forming an oversew suture at the second end of prosthesis—graft or stent-graft 13 (FIG. 12).

Then, if necessary, cartridges 9 of second working head 7 are reloaded with standby U-shaped staples 11. This is accomplished by partial removal of flexible pushers 51 activated, in their turn, via second pressure bush 41, fourth connecting means—pressure tube 137 and second control means—pressure handle 131 of second module 19 of control mechanism 15, which are associated with these pushers. As a result, each spring-loaded U-shaped staple 11 emerges from through slot 55 of corresponding cartridges 9, and then, at reverse motion of a corresponding flexible pusher 51, moves by its action into recess 61, wherein the basic U-shaped staple 11 was located before. Now second head 7 of apparatus 1 for delivery and fixation is ready for another suturing.

If another suturing is necessary, standby U-shaped staples 11 in cartridges 9 of second working head 7 are set in motion. This is accomplished by action of flexible pushers 51 activated, in their turn, via second pressure bush 41, fourth connecting means—pressure tube 137 and second control means—pressure handle 131 of second module 19 of control mechanism 15, which are associated with these pushers. As a result, pointed ends 57 of each standby U-shaped staple 11 enter forming grooves 71 of curvilinear guiding surface 69 of a corresponding removable cartridge 9, are curled therein, diverging in opposite directions from one another, pierce the wall of the second end of prosthesis—graft or stent-graft 13 and surrounding wall of aorta 200 (FIG. 12) and return again over a spiral to curvilinear guiding surface 69, repeating this rotation if permitted by the given length of staples 11. Thereby the wall of the second end of prosthesis—graft or stent-graft 13 gets sutured again by wire spirals formed from standby U-shaped staples 11, to a corresponding wall area of aorta 200, and standby U-shaped staples 11 themselves entirely emerge from recesses of corresponding cartridges 9 and are released from these cartridges 9, forming a second oversew suture at the second end of prosthesis—graft or stent-graft 13 (FIG. 12).

Then the second end of a delivered and sutured prosthesis—graft or stent-graft 13 is separated from apparatus 1 for delivery and fixation, both its expandable working heads 5 and 7 are returned in initial position, this apparatus 1 is set in inoperative position and removed from prosthesis—graft or stent-graft 13 and from the blood vessel—aorta 200.

The application of proposed apparatus 1, as well as realization on the basis of this apparatus of a new and improved method for delivery and fixation of prosthesis—grafts or stent-grafts 13 to the wall of a blood vessel—aorta 200 from within the latter allows, in the authors' opinion, to solve the problem of endoscopic delivery and endoscopic suturing of grafts or stent-grafts 13 to aorta 200 without performing a cavitary operation. Simultaneously, it solves the problem of preventing a sutured graft or stent-graft 13 from shifting from the required position within the blood vessel—aorta 200, which may occur due to peristelsic oscillations of walls of aorta 200 and shakings of a patient's body during its movement.

While this invention has been described in conjunction with specific embodiment thereof, it is evident that many alternatives, modifications and variations will be apparent to those skilled in the art. Accordingly, the preferred embodiments of the invention as set forth herein are intended to be illustrative, not limiting. Various changes may be made without departing from the spirit and scope of the invention as defined in the following claims.

1. An apparatus for delivery and double-ended fixation of endovascular grafts or stent-grafts, comprising:
a) a tubular body configured for positioning within a vessel, this tubular body being rigid in longitudinal direction and flexible in lateral direction, at one its end there are located two working heads, and at the other end—a control mechanism;
b) a first working head with cartridges intended for location of fastener means, this working head is rigidly secured at the free end of tubular body and is made expandable to keep the first end of a delivered graft in contact with the inner surface of a blood vessel at the moment of their mutual fixation;
c) a second working head with cartridges intended for location of fastener means, this working head is secured near the free end of tubular body, some distance away from the first working head, aligned with the latter, this working head is also made expandable and serves to keep the second end of the delivered graft in contact with the inner surface of a blood vessel at the moment of their mutual fixation;
d) two cartridge groups, each including eight cartridges, which are incorporated in the construction of first and second working heads respectively, all cartridges are unified and interchangeable and each of the cartridges is intended for location at least of two fastener means, and all cartridges are provided with means for retaining therein fastener means, for imparting thereto progressive motion, for forming fastener means during their extension from these cartridges, as well as for storing and feeding standby fastener means;
e) a control mechanism, including a first control module for operation of the first working head, which is located at the free end of tubular body opposite to this head, as well as a second control module located on the tubular body between the first control module and second working head, rigidly connected with this second working head and serving for its operation, the first and second control modules being joined together via a connecting means,
whereby a surgical operation may be performed for graft or stent-graft delivery and fixing both its ends to the walls of a blood vessel wall, substantially the aorta, from inside the aorta.

2. The apparatus for delivery and double-ended fixation of endovascular grafts according of claim 1, wherein
f) said second working head is secured near the free end of tubular body, some distance away from the first working head, aligned with the latter and capable of reciprocation relative thereto, and
g) a connecting means located on said tubular body between the first and second control modules and capable of providing both rigid fixation of modules relative to one another, or reciprocation of second control module and second working head rigidly attached thereto relative to the first control module,
whereby a surgical operation may be performed for graft delivery, spreading within the blood vessel and fixing both its ends to the walls of a blood vessel wall, substantially the aorta, from inside the aorta.

3. The apparatus of claim 1, wherein said first expandable working head contains a first pressure bush and first and second bearing bushes, which are arranged in succession one after another beginning from the free end of this working head and in alignment with said tubular body, as well as a first axial tube rigidly connected with first bearing bush and passing via a through axial hole in second bearing bush.

4. The apparatus of claim 3, wherein said first expandable working head is provided at least with eight cartridges, pivotally mounted by one their ends on said first bearing bush and pivotally connected by their other ends with bearing levers pivotally mounted on said second bearing bush.

5. The apparatus of claim 1, wherein said second working head located some distance away from first expandable working head contains a third and fourth bearing bushes and second pressure bush, which are arranged in succession one after another beginning from the free end of this working head facing the first expandable working head, as well as a second axial tube rigidly connected with third bearing bush and passing via through axial holes in fourth bearing bush and in second pressure bush, said third and fourth bearing bushes and second pressure bush being aligned with said tubular body.

6. The apparatus of claim 5, wherein said second working head is provided at least with eight cartridges, secured by one their ends on said fourth bearing bush and pivotally connected by their other ends with bearing levers pivotally mounted on third bearing bush.

7. The apparatus of claim 4 and 6, wherein said first and second working heads are provided with means for securing a delivered graft located on bearing levers.

8. The apparatus of claim 7, wherein said means for securing a delivered graft are shaped substantially as radial tongues located at one of the ends of corresponding bearing levers, near their pivotal connections with corresponding cartridges.

9. The apparatus of claim 1, wherein each of said cartridges contains a means for retaining therein at least one basic fastener means, substantially U-shaped staple, this means for retaining being located along the cartridge and contains substantially a sliding lid.

10. The apparatus of claim 9, wherein each of said cartridges also contains at least one means for imparting progressive motion to said fastener means, substantially U-shaped staple, and this means contains a flexible pusher, rigid in longitudinal direction and flexible in lateral direction.

11. The apparatus of claim 10, wherein each of said cartridges is provided with a flexible pusher, which is associated by one its end with first or second pressure bush of first or second expandable working head respectively and located under said sliding lid to reciprocate in the clearance between the cartridge bottom and said sliding lid.

12. The apparatus of claim 9, wherein each of said cartridges also contains a means for forming fastener means, which is located in this cartridge near one of its ends, as well as at least one basic fastener means, substantially U-shaped staple, located in this cartridge substantially along its longitudinal axis, with free pointed ends facing said means for forming, the U-shaped staple being located in the cartridge so that it is capable of progressively extending therefrom and being formed during this extension.

13. The apparatus of claim 11, wherein each of said cartridges has a bottom and sliding lid and contains a means for storing at least one standby fastener means, substantially U-shaped staple, this means for storing contains a slot in the cartridge bottom, wherein the standby U-shaped staple is disposed substantially along the cartridge longitudinal axis, with free pointed ends facing said means for forming, the U-shaped staple being located in the bottom slot of this cartridge substantially under said flexible pusher and capable of extending from this slot by action of a spring and at partial removal of the flexible pusher from the clearance between the cartridge bottom and said sliding lid.

14. The apparatus of claim 13, wherein said slot in the bottom of each of said cartridges is a through one, and the standby U-shaped staple is located in this slot immediately under said flexible pusher and spring-loaded on the opposite side.

15. The apparatus of claim 13, wherein each of said cartridges has a body with a bottom provided with a recess for location at least of one basic fastener means, substantially U-shaped staple, and a through slot for location at least of one standby fastener means, substantially standby U-shaped staple, slots for mounting said sliding lid, as well as a longitudinal duct for location at least of one said flexible pusher, the basic U-shaped staple being located in said bottom recess of the cartridge body in frictional contact with the sliding lid, which prevents its dropping out of this recess, and the standby spring-loaded U-shaped staple being located in the through bottom slot of the cartridge body in frictional contact with the flexible pusher, which prevents its extension from this through slot before partial removal of this flexible pusher.

16. The cartridge of claim 15, which has a body with a bottom provided with a recess at least for one basic fastener means, substantially U-shaped staple, this recess is symmetric about the longitudinal axis of this cartridge and parallel with its outer surface, the cartridge being provided with a means for forming said U-shaped staple during its extension from said recess, and this means contains a curvilinear guiding surface connecting the bottom of said recess for U-shaped staple with the cartridge outer surface.

17. The cartridge of claim 16, which is provided with a means for forming the U-shaped staple during its extension from this cartridge, this means containing a curvilinear guiding surface connecting the bottom of said recess for the U-shaped staple with the cartridge outer surface, said curvilinear guiding surface provided with forming grooves.

18. The cartridge of claim 17, wherein said forming grooves diverge at an acute angle from one another and from the cartridge longitudinal axis.

19. The cartridge of claim 18, wherein at least one basic fastener means, substantially U-shaped staple, has pointed free ends and is located in said body recess of this cartridge in such a way, that its pointed ends face corresponding forming grooves on the curvilinear guiding surface.

20. The cartridge of claim 18, wherein said forming grooves diverge at an acute angle from one another and from the cartridge longitudinal axis, which provides helical oppositely directed curling of pointed ends of a corresponding U-shaped staple during its motion over the body recess and forming grooves of the cartridge curvilinear guiding surface.

21. The cartridge of claim 1, shaped substantially as a polyhedral prism with two skewed side faces inclined at an acute angle to one another, the vertex of this acute angle lying on the longitudinal axis of the expandable working head.

22. The cartridge of claim 21, shaped substantially as a polyhedral prism provided with means for connection with the first or fourth bearing bushes of first or second working head or with a corresponding bearing lever, and these means substantially contain arms with holes, which project from both bases of said polyhedral prism and are integral with this polyhedral prism.

23. The apparatus of claim 1, wherein each of said cartridges contains at least one basic and at least one standby fastener means, substantially U-shaped staples, and these staples are made from one of the materials of a group including stainless steel, titanium and shape memory alloys.

24. The apparatus of claim 1, which is additionally provided with a means for axial precision positioning containing a measuring scale on the surface of said tubular body.

25. The apparatus of claim 1, which is additionally provided with means for radial precision positioning containing markers—X-ray marks regularly applied on the outer surface of said cartridges and serving for orientation of the apparatus working heads by their operation angle.

26. The apparatus of claim 1, wherein said control mechanism is provided at least with one first control module and at least with one second control module joined together via a connecting means, the first control module being located at the free end of tubular body opposite to the first expandable working head, and said second control module being rigidly connected with the second expandable working head and located on this tubular body near the first control module, between the latter and said second expandable working head.

27. The apparatus of claim 26, wherein said connecting means located on the tubular body between first and second control modules, and providing both their mutual rigid fixation, or reciprocation of second control module and second working head rigidly attached thereto relative to the first control module, substantially contains a holder with a gear pivotally mounted therein and provided with a lock and rotary drive, as well as two toothed racks functionally engaged with this gear, the first of said racks being rigidly connected with first control module, and the second rigidly connected with second control module.

28. The apparatus of claim 27, wherein said connecting means located on the tubular body between said first and second control modules is reciprocable along the axis of this tubular body and additionally supplied with a handle and support for carrying the apparatus and its location on an operation table.

29. The apparatus of claim 27, wherein said connecting means located on the tubular body between said first and second control modules is provided with a gear rotary drive, substantially mechanic or electric.

30. The apparatus of claim 26, wherein said first control module of the control mechanism contains a hollow body having a first and second ends, as well as a reciprocable slider enclosed therein, the slider being provided with a holding handle rigidly attached thereto, functionally associated with a pivotal head located at the second end of this hollow body and forming together with this pivotal head the first control means of first control module of the control mechanism.

31. The apparatus of claim 30, wherein said first control module of the control mechanism also contains a first pressure handle serving as the second control means of first control module of the control mechanism, this first pressure handle is pivotally attached to said holding handle and has a short free end enclosed in said slider, the slider being rigidly connected with the first axial tube serving as a first connecting means, and the short free end of said pressure handle is functionally associated with a tie serving as a second connecting means of first control module of the control mechanism.

32. The apparatus of claim 31, wherein said first pressure handle is provided with a swing lock pivotally secured on said slider, as well as with a flat return spring secured by one its end to this pressure handle, and by its second hand to said holding handle.

33. The apparatus of claim 31, wherein said first bearing bush is connected via first connecting means with said first control means of first control module of the control mechanism, first pressure bush is functionally connected via second connecting means with said second control means of first control module of the control mechanism, and second bearing bush is rigidly connected with a distance tube located between first and second working heads, said first connecting means and said second connecting means being enclosed in said distance tube and tubular body concentrically with one another, said distance tube and this tubular body.

34. The apparatus of claim 26, wherein said second control module of the control mechanism contains a hollow body with a handle rigidly attached thereto, this body has a first and second ends, is rigidly connected by its first end via a retaining tube with the fourth bearing bush of said second working head and also includes a slider reciprocally enclosed in the body, the slider being functionally associated with the pivotal head located at the second end of this hollow body and forming together with this pivotal head the first control means of second control module of the control mechanism.

35. The apparatus of claim 34, wherein the second control module also has a second pressure handle serving as its second control means and pivotally attached to said handle or to the hollow body, this second pressure handle has a short free end enclosed in said slider, the slider being rigidly connected with a tubular tie serving as a third connecting means, and the short free end of said pressure handle functionally associated with a pressure tube serving as the fourth connecting means of the control mechanism and rigidly connected with said second pressure bush.

36. The apparatus of claim 35, wherein said second pressure handle is provided with a swing lock pivotally mounted in said hollow body, as well as a flat return spring secured by one its end to this second pressure handle, and by its second end to said hollow body handle.

37. The apparatus of claim 35, wherein said third bearing bush is connected via third connecting means with said first control means of second control module of the control mechanism, second pressure bush is functionary connected via fourth connecting means with said second control means of second control module of the control mechanism, and fourth bearing bush is connected with the body of second control module via retaining tube, said third connecting means of second module is enclosed in said retaining tube, and said fourth connecting means of second module is located outside this retaining tube and they are all arranged concentrically with one another and this retaining tube.

38. The apparatus of claim 33 and 37, wherein said first and second connecting means linking the first working head with first control module of the control mechanism are arranged concentrically with one another within said third connecting means, retaining tube and fourth connecting means, which are also arranged concentrically with one another and connect second working head with second control module of the control mechanism, said distance tube being located between first and second working heads outside the first and second connecting means and aligned with said third and fourth connecting means.

39. The apparatus of claim 38, wherein said fourth connecting means located outside, concentrically with said retaining tube, third connecting means, as well as with first and second connecting means, forms a tubular body configured for positioning within a vessel, and this tubular body, together with connecting means enclosed therein, is rigid in longitudinal direction and flexible in lateral direction.

40. A method for delivery and double-ended fixation of a prosthesis, the graft or stent-graft, to the wall of a blood vessel from inside the latter, comprising the following successive steps:
i) preparing the prosthesis for delivery, mounting and securing its both ends on the apparatus working heads, as well as locating the prosthesis between working heads and its crimping to a given outer diameter;
ii) setting the apparatus for delivery and fixation in operative position, inserting it in a corresponding blood vessel, bringing said first expandable working head to the securing area, precision axial and radial positioning in a given area of the blood vessel via a measuring scale on the surface of tubular body and via markers—X-ray contrast marks, as well as deploying the first expandable working head in such a way, that the first end of a delivered prosthesis is in contact with the inner surface of the blood vessel at the moment of their mutual fixation, and the cartridges of this expandable working head are located near corresponding suturing points;
iii) setting in motion said U-shaped staples of the cartridge group of first expandable working head accomplished by action of said flexible pushers activated, in their turn, via first pressure bush, second connecting means and second control means of first module of the control mechanism, which are associated with these pushers, whereby pointed ends of each of the U-shaped staples enter forming grooves of curvilinear guiding surface of a corresponding removable cartridge, are curled therein, diverging in opposite directions from one another, pierce the prosthesis wall and surrounding blood vessel wall and return again over a spiral to said curvilinear guiding surface, repeating this rotation if permitted by the given staple length, whereby the wall of the prosthesis first end gets sutured by wire spirals formed from U-shaped staples, to a corresponding wall area of the blood vessel, and said U-shaped staples entirely emerge from recesses of corresponding cartridges and are released from these cartridges, forming an oversew suture at the first end of the prosthesis;
iv) reloading the cartridge group of first expandable working head with standby U-shaped staples accomplished by partial removal of said flexible pushers activated, in their turn, via first pressure bush, second connecting means and second control means of first module of the control mechanism, which are associated with these pushers, whereby each of the spring-loaded standby U-shaped staples emerges from the through slot of a corresponding cartridge, and then, at reverse motion of a corresponding flexible pusher, moves by its action into said recess, where the basic U-shaped staple was located before, whereby first working head of the apparatus is made ready for another suturing;
v) if another suturing is necessary, setting in motion said standby U-shaped staples of first expandable working head accomplished by action of said flexible pushers activated, in their turn, via first pressure bush, second connecting means and second control means of first module of the control mechanism, which are associated with these pushers, whereby pointed ends of each of the standby U-shaped staples enter the forming grooves of curvilinear guiding surface of a corresponding removable cartridge, are curled therein, pierce the wall of the prosthesis first end and surrounding blood vessel wall and return again over a spiral to said curvilinear guiding surface, repeating this rotation if permitted by the given staple length, whereby the wall of the prosthesis first end gets sutured again by wire spirals formed from standby U-shaped staples, to a corresponding area of the blood vessel wall, and said standby U-shaped staples entirely emerge from recesses of corresponding cartridges and are released from these cartridges, forming a second oversew suture at the first end of this prosthesis;
vi) spreading, if it is necessary, the prosthesis sutured at one end accomplished by turning the gear of said connecting means, which enables the second control module and second working head rigidly connected therewith to move along the apparatus tubular body some predesigned distance towards the first control module;
vii) setting in motion said U-shaped staples of the cartridge group of second working head accomplished by action of said flexible pushers activated, in their turn, via second pressure bush, fourth connecting means and second control means of second module of the control mechanism, which are associated with these pushers, whereby pointed ends of each of the U-shaped staples enter the forming grooves of curvilinear guiding surface of a corresponding removable cartridge, are curled therein, diverging in opposite directions from one another, pierce the prosthesis wall and surrounding blood vessel wall and return again over a spiral to said curvilinear guiding surface, repeating this rotation if permitted by the given staple length, whereby the wall of the prosthesis second end gets sutured by wire spirals formed from U-shaped staples, to a corresponding area of the blood vessel wall, and said U-shaped staples entirely emerge from recesses of corresponding cartridges and are released from these cartridges, forming an oversew suture at the second end of this prosthesis;
viii) reloading the cartridge group of second working head with standby U-shaped staples accomplished by partial removal of said flexible pushers activated, in their turn, via second pressure bush, fourth connecting means and second control means of second module of the control mechanism, which are associated with these pushers, whereby each of the spring-loaded standby U-shaped staples emerges from the through slot of a corresponding cartridge, and then, at reverse motion of a corresponding flexible pusher, moves by its action into said recess, where the basic U-shaped staple was located before, whereby the second working head of said apparatus for delivery and fixation is made ready for another suturing;
ix) if another suturing is necessary, setting in motion said standby U-shaped staples in cartridges of second working head accomplished by action of said flexible pushers activated, in their turn, via second pressure bush, fourth connecting means and second control means of second module of the control mechanism, which are associated with these pushers, whereby pointed ends of each of the standby U-shaped staples enter the forming grooves of curvilinear guiding surface of a corresponding removable cartridge, are curled therein, diverging in opposite directions from one another, pierce the wall of said prosthesis second end and surrounding blood vessel wall and return again over a spiral to said curvilinear guiding surface, repeating this rotation if permitted by the given staple length, whereby the wall of this prosthesis second end gets sutured again by wire spirals formed from standby U-shaped staples, to a corresponding area of this blood vessel wall, and said standby U-shaped staples entirely emerge from recesses of corresponding cartridges and are released from these cartridges, forming a second oversew suture at the second end of a corresponding prosthesis;
x) separating both ends of a delivered and sutured prosthesis from the apparatus for delivery and fixation, returning both its expandable heads to original position;
xi) setting the apparatus for delivery and fixation in inoperative position and its removal from the prosthesis and from the blood vessel.
